(19) United States
(12) Patent Application Publication
(10) Pub. No.: US 2020/0297629 A1
(43) Pub. Date: Sep. 24, 2020

(54) EMOLLIENT EMULSION

(71) Applicant: 3J's Green Equine Care, LLC, Fallbrook, CA (US)

(72) Inventor: Janice Simmons, Huntington Beach, CA (US)

(73) Assignee: 3J's Green Equine Care, LLC, Fallbrook, CA (US)

(21) Appl. No.: 16/894,851

(22) Filed: Jun. 7, 2020

Related U.S. Application Data
(60) Provisional application No. 62/868,899, filed on Jun. 29, 2019.

(51) Int. Cl. A61K 9/107; A61K 9/12; A61K 47/44; A61K 36/28; A61K 47/14; A61K 47/02; A61K 31/05; A61D 7/00

(52) U.S. Cl. CPC A61K 9/107; A61K 9/12; A61K 47/44; A61D 7/00; A61K 47/14; A61K 47/02; A61K 31/05; A61K 36/28

(57) ABSTRACT

A composition of matter with emollient properties. The composition takes the physical form of an oil base homogenized into water, making it suitable for dispensation in large quantities with a spray bottle or a continuous flow. Preparation begins with refinement of MCT coconut oil to remove lauric acid. The coconut oil is mixed with arnica oil, castor oil, CBD oil, and sulfur to form the oil base. Ideal proportions for the oil base are approximately 8:39:10:40:3 arnica:castor:CBD:coconut:sulfur. The oil base is then homogenized into alkaline water. Ideal parameters are approximately 11.5 pH water and a ratio of approximately 7:93 oil:water. Suitable variations on the ideal parameters are disclosed below.

### 1. FIELD OF THE INVENTION

This invention is in the field of emulsions, specifically spray-dispensed emulsions with emollient properties.

### 2. BACKGROUND OF THE INVENTION

Pets, horses, and barn animals often experience dry or irritated skin. Emollient (skin-soothing) products, for humans and pets alike, come in three common physical forms: creams, liquid elixirs dispensed from droppers, and aerosol oil sprays.

Cream products present difficulties and inefficiencies, especially on animals. A groomer will apply the cream by hand. It is difficult to penetrate the animal's hair and get the cream directly onto the skin. The only way to effectively apply this kind of product is to part the fur and directly touch the skin. When the skin is irritated, this can cause the animal pain, discomfort, or further irritation.

A second form of skin treatment is a liquid dropper. While this can be effective for topical treatment of one small pet, it is not acceptable for treatment of large animals like horses or other barn animals, let alone multiple animals.

A third form of treatment is an oil spray. The problem with 100% oil sprays is that they do not penetrate the coat well. They do not disperse on the surface as well as water, tending to stick to the outer coat instead. In the case of severe skin irritation, the pet must often be shaved before treatment is applied.

Hemp oil, aka cannabidiol or CBD oil, is an increasingly common plant oil used for emollients and a variety of other products. It is derived from the Cannabis sativa plant. CBD oil contains less than 3% THC, the psychoactive compound in marijuana. Hemp-based CBD oil is legal under federal law and is permitted in every state.

What is needed is an emollient product, especially a CBD-based emollient, homogenized in water. The emulsion with water will make the product easy to dispense in large quantities. A water-based mixture is also more effective for penetrating fur and treating skin. The need for this product necessitates a method of making it.

### 3. DESCRIPTION OF RELATED TECHNOLOGY

The most common natural ingredients used for emollient products, whether for human or animal care, are plant oils and mineral oils. The hemp-based skin-care products available on the market today, particularly for pets, are composed entirely of oils. Some come in the form of a cream, such as Coat Defense Trouble Spot Drying Paste. Shanti Salve and Coat Defense Trouble Spot Drying Paste are other examples of CBD-based emollient creams, the latter intended for use on horses. Other products currently on the market are viscous liquids dispensed by dropper, such as Clean Pet Nutrition Premium Hemp Oil. One example of a CBD spray bottle for pet skin care is the “AndPets CBD Hot Spot Spray” manufactured by AndHemp.

Common synthetic ingredients include triglycerides, benzoates, myristates, palmitates, and stearates. Products made entirely from natural ingredients are appealing to many consumers today. The present invention uses all-natural active ingredients, namely arnica oil, castor oil, CBD oil (discussed above in the Background of the Invention), sulfur, and alkaline water.

Arnica is a genus of wildflower. Arnica oil, especially from Arnica montana, is commonly used in herbal drug products for inflammatory conditions and muscle pain.

Castor oil is derived from the seeds of the Ricinus communis plant. Rich in fatty acids, it has been used as an elixir for beautiful hair and supple skin. It can also assist in fighting many skin conditions such as scars, fungal disease, dry spots, and itchiness.

Sulfur has a long history of use for a variety of dermatological disorders, as an ingredient in acne ointments, in anti-dandruff shampoos, and wound tinctures. Aside from elemental sulfur, compounds such as MSM (Methylsulfonyl Methane) or DMSO (Dimethyl Sulfoxide) are common in emollient products.

Finley (WIPO patent application WO2016123475A1) discloses “cannabis oil extracts and compositions thereof.” Each extract is made with the four-step process of: (1) eluting cannabinoids from cannabis plant material with a solvent to produce an eluate; (2) filtering the eluate with a filter to produce a filtrate; (3) evaporating the solvent from the filtrate with a distiller to produce a distillate; and (4) dehydrating/purging the distillate with a dehydrator or vacuum oven to prepare the extract.

The “compositions” in the Finley application consist of the extract as well as “vitamin E, an essential oil, an herbal extract, a probiotic, a homeopathic remedy, a flower essence, [and/or] a carrier oil.” Examples of these additional ingredients are provided in the form of encyclopedic lists that include arnica, castor oil, sulfur, and coconut oil. (See Finley paragraphs 150-154). The application does not single out any combination of ingredients from these candidate lists, i.e. it does not specifically disclose a combination of arnica, castor oil, CBD, and sulfur. The relative abundances of ingredients in the Finley specification are likewise too broad to constitute a prior art disclosure, with ranges like “a carrier oil will typically be present in an amount ranging from about 1% to about 95%.”

Another popular class of skin-care product is micellar water. It consists of micelles, or molecular oil droplets, suspended in water. Some of today's leading micellar water products are manufactured by Gamier, L'Oreal, and Bioderma. These products all contain disodium EDTA and at least one glycol/glycerin/glyceride ingredient. Otherwise, they have diverse ingredient lists.

The present invention makes use of alkaline water. Electrolysis involves running a current through a body of water, with a positively charged cathode on one end and a negatively charged anode on the other end. The current assists in ionizing the water into H+ and OH− ions. The H+ ions, hence the acidic water, gathers toward the anode and the OH− ions, hence the alkaline water, gathers at the cathode. The alkaline water can then be isolated and drawn out of the electrolysis chamber. Popularly called “ionized water”, it is often sought as a remedy for skin conditions as well as for other benefits such as burning fat.

### 4. SUMMARY OF THE INVENTION

The present invention is a skin-care product, ideally for pets and cattle because of its unique ability to penetrate body hair. Chemically, its active ingredients are CBD oil and other emollients such as castor oil, arnica, and optionally sulfur. The active ingredients are merged into ultra-refined coconut oil to form a concentrate that shall be called the oil base. The oil base is then homogenized in alkaline water. The finished product is an emulsion dispensed from a spray bottle.

### 5. BRIEF DESCRIPTION OF THE DRAWINGS

Drawings are not necessary to understand this invention.

### 6. DETAILED DESCRIPTION OF THE PREFERRED EMBODIMENTS

The proper preparation of this product requires medium-chain triglycerides (MCT). Coconut oil has the advantage of being a fat heavy in MCT. To achieve the purposes of the present invention, my experimentation has shown that the coconut oil must be refined to remove the lauric acid. Using coconut oil without lauric acid is the first key step in preparing the invention.

Next, the active ingredients must be mixed into the refined coconut oil. The optimal proportion of ingredients is approximately 40% refined coconut oil as described in the previous paragraph, 30-40% castor oil, 10% CBD oil, up to 8% arnica oil, up to 6% sulfur, and a trace amount, roughly 0.01%, of lavender oil or another essential oil. For best results, the CBD oil should be at least 82% strength, and it should be properly processed and certified analyzed before being used as an ingredient. All oil ingredients are heated and stirred for thorough mixing. This mixture is called the “oil base”.

The next step in preparation is emulsion in highly alkaline ionized water. A pH of at least 11.5 works best. The oil base and the alkaline water are combined ideally in the ratio of about 93% alkaline water and 7% oil base. This will result in a final product that is approximately 93% alkaline water, 2.8% coconut oil, 2.1-2.8% castor oil, 0.7% CBD oil, up to 0.56% arnica oil, up to 0.42% sulfur, and roughly 0.0007% essential oil. Embodiments of the invention with these approximate proportions and acceptable margins of error are described and claimed below. We describe “low sulfur” and “high sulfur” versions. A lower sulfur content is more appropriate for animal treatment, and a higher sulfur content is a more suitable adaptation for human treatment.

Finally, the mixture is blended for ten minutes to ensure full homogenization. Alkaline water promotes the breakdown of fatty acids much more effectively than neutral water. The product will appear milky white when it is properly mixed.

A method of making the product, comprising the steps of forming the oil base and then homogenizing it into the alkaline water, are described and claimed below.

I claim:

1. A homogenized emulsion comprising, in percentages by weight of whole solution,
1.4% to 4.3% medium-chain-triglyceride carrier oil;
1.3% to 4.3% castor oil;
0.4% to 1.1% CBD oil at minimum 80% strength;
0.28% to 0.85% arnica oil;
0% to 0.32% sulfur;
with the remainder of the solution being alkaline water in a pH range of 11.0 to 12.0.

2. The emulsion of claim 1, wherein the percentages by weight of the whole solution are more specifically:
1.4% to 4.3% for castor oil;
0% to 0.04% for sulfur.

3. The emulsion of claim 2, further comprising 0.0004% to 0.0011% essential oil.

4. The emulsion of claim 1, wherein the percentages by weight of the whole solution are more specifically:
1.3% to 4.0% for castor oil;
0.11% to 0.32% for sulfur.

5. The emulsion of claim 4, further comprising 0.0004% to 0.0011% essential oil.

6. The emulsion of claim 1, wherein the percentages by weight of the whole solution are more specifically:
2.1% to 3.6% medium-chain-triglyceride carrier oil;
2.0% to 3.6% for castor oil;
0.53% to 0.89% for CBD oil;
0.43% to 0.71% for arnica oil;
0.02% to 0.26% for sulfur.

7. The emulsion of claim 6, wherein the percentages by weight of the whole solution are more specifically:
2.1% to 3.6% for castor oil;
0% to 0.04% for sulfur.

8. The emulsion of claim 7, further comprising 0.0005% to 0.0009% essential oil.

9. The emulsion of claim 6, wherein the percentages by weight of the whole solution are more specifically:
2.0% to 3.3% for castor oil;
0.16% to 0.26% for sulfur.

10. The emulsion of claim 9, further comprising 0.0005% to 0.0009% essential oil.

11. A method of making an emulsion, comprising the steps of
providing a medium-chain-triglyceride carrier oil, which naturally includes lauric acid;
refining the carrier oil to remove the lauric acid;
providing CBD oil at minimum 80% strength;
providing castor oil, arnica oil, and sulfur;
combining the preceding provided ingredients to form an oil base, composed by weight of 19%-61% castor oil, 5.1%-15.2% CBD oil, 4.1%-12.2% arnica oil, and 0.2%-4.5% sulfur, with the remainder of the oil base being the refined medium-chain-triglyceride carrier oil;
heating the oil base to form a homogeneous mixture;
providing alkaline water with a pH level between 11 and 12;
combining the oil base and the alkaline water in the amounts of 5% to 10% oil base and 90% to 95% alkaline water by weight;
blending the oil base/alkaline water mixture for ten minutes to homogenize the mixture into an emulsion.

12. The method of claim 11, wherein the percentages by weight of the oil base are more specifically:
20% to 61% for castor oil;
0% to 0.6% for sulfur.

13. The method of claim 12, further comprising the step of adding essential oil to the oil base in the amount of 0.01% to 0.02% by weight.

14. The method of claim 11, wherein the percentages by weight of the whole solution are more specifically:
1.3% to 4.0% for castor oil;
0.11 to 0.32% for sulfur.

15. The method of claim 14, further comprising the step of adding essential oil to the oil base in the amount of 0.01% to 0.02% by weight.

16. The method of claim 11, wherein the percentages by weight of the oil base are more specifically:
29% to 51% for castor oil;
7.6% to 12.7% for CBD oil;
6.1% to 10.2% for arnica oil;
0.3% to 3.8% for sulfur.

17. The method of claim 16, wherein the percentages by weight of the oil base are more specifically:
30% to 51% for castor oil;
0% to 0.5% for sulfur.

18. The method of claim 17, further comprising the step of adding essential oil to the oil base in the amount of 0.008%-0.013% by weight.

19. The method of claim 16, wherein the percentages by weight of the oil base are more specifically:
29% to 48% for castor oil;
2.3% to 3.8% for sulfur.

20. The method of claim 19, further comprising the step of adding essential oil to the oil base in the amount of 0.008%-0.013% by weight.
